(19) United States
(12) Patent Application Publication
(10) Pub. No.: US 2025/0179408 A1
(43) Pub. Date: Jun. 5, 2025

(54) HYPERBARIC INCUBATION SYSTEM AND METHOD

(71) Applicant: ALVÉOCELL. INC., Quebec City (CA)

(72) Inventor: Jacquelin BRETON, Quebec City (CA)

(21) Appl. No.: 18/524,872

(22) Filed: Nov. 30, 2023

(51) Int. Cl. C12M 1/00; C12M 1/02; C12M 1/34

(52) U.S. Cl. CPC C12M 41/14; C12M 29/04; C12M 41/18; C12M 41/26; C12M 41/34; C12M 41/40

(57) ABSTRACT

The present disclosure provides systems and methods for incubation of cell cultures. The system can include a compressor for providing airflow at atmospheric or hyperbaric conditions, a reservoir, an incubator for housing cell cultures that produce CO₂ enriched airflow, an exhaust container, and an exhaust pump in the exhaust container and in fluid communication with the incubator for drawing CO₂ enriched airflow out of the incubator to maintain a stable pH level for the cell culture.

### TECHNICAL FIELD

The technical field generally relates to an incubation system and method for incubating cell cultures, and more particularly to a multizone incubation system operable at hyperbaric conditions.

### BACKGROUND

Cell culture incubators are units configured to control a desired temperature, humidity, or other parameters within the unit to allow cell cultures placed therein to grow. However, there are various challenges to providing hyperbaric conditions for cell culture incubation, even though such hyperbaric conditions could be of interest. There is a need for a technology that facilitates cell culture growth in hyperbaric conditions.

### SUMMARY

Various aspects of the systems and methods for the incubation of cell cultures are described herein. Hyperbaric incubation can leverage certain features such as providing chambers as well as air displacement components such that air is fed through the chambers establishing hyperbaric pressure conditions while removing CO₂ generated by the cell culture.

In accordance with one aspect, there is provided a hyperbaric incubation system comprising a compressor for providing pressurized air; a hyperbaric chamber comprising a hyperbaric chamber wall defining a hyperbaric chamber cavity; a hyperbaric chamber inlet defined in the wall for receiving the pressurized air from the compressor; a hyperbaric chamber pressure relief open port defined in the wall, wherein closing the hyperbaric chamber pressure relief open port allows hyperbaric chamber pressurization and opening the hyperbaric chamber pressure relief open port allows hyperbaric chamber depressurization; and a hyperbaric chamber door provided in the hyperbaric chamber wall and having an open position for allowing access to the hyperbaric chamber cavity and a closed position for providing hyperbaric conditions; a reservoir located within the hyperbaric chamber cavity and comprising a reservoir wall defining a reservoir cavity; a reservoir inlet defined in the reservoir wall and being in fluid communication with the hyperbaric chamber cavity for receiving the pressurized air therefrom; and a reservoir outlet defined in the reservoir wall; an incubator located within the hyperbaric chamber cavity and comprising an incubator wall defining an incubator cavity, and configured to receive at least one cell culture container therein, wherein the at least one cell culture container is configured to contain a cell culture which produces CO₂ that becomes part of the pressurized air to create CO₂ enriched pressurized air; an incubator inlet defined in the incubator wall and being in fluid communication with the reservoir outlet for receiving the pressurized air therefrom; an incubator outlet defined in the incubator wall; and an incubator door provided in the incubator wall and having an open position for allowing access to the incubator cavity and a closed position for providing hyperbaric conditions; an exhaust container located within the hyperbaric chamber cavity and comprising an exhaust container wall defining an exhaust container cavity; an exhaust container inlet defined in the exhaust container wall and being in fluid communication with the reservoir outlet for receiving the pressurized air therefrom; and an exhaust container outlet defined in the exhaust container wall and being in fluid communication with the hyperbaric chamber cavity; and an exhaust pump in fluid communication with the exhaust container inlet for drawing the CO₂ enriched pressurized air out of the incubator to maintain a stable pH for the cell culture.

In some implementations, the pressure of the hyperbaric incubation system is within a range of 1.2 atm to 1.5 atm. In some implementations, the system further includes a first heat exchanger downstream of the compressor and configured to reduce a temperature of the pressurized air. In some implementations, the system includes a second heat exchanger downstream of the compressor and configured to reduce or increase a temperature the pressurized air. In some implementations, the second heat exchanger is a Peltier heat exchanger. In some implementations, the system includes at least one filter downstream of the compressor wherein the pressurized air is filtered prior to air entering the hyperbaric chamber. In some implementations, the at least one filter comprises a carbon filter. In some implementations, the at least one filter comprises a HEPA filter. In some implementations, the at least one cell culture container comprises: a cell culture container inlet; and a cell culture container outlet, wherein at least one inlet tube connects the cell culture container inlet to the incubator inlet and at least one outlet tube connects the cell culture container outlet to the incubator outlet. In some implementations, the reservoir comprises at least one reservoir open port provided on the wall of the reservoir, the exhaust container comprises at least one exhaust open port provided on the wall of the exhaust container, the incubator comprises: at least a first incubator open port being in fluid communication with the at least one reservoir open port; and at least a second incubator open port being in fluid communication with the at least one exhaust container open port. In some implementations, each of the reservoir, exhaust container and incubator open ports are configured to actuate in response to a difference in pressure between the reservoir, the exhaust container, and the incubator. In some implementations, each of the reservoir, exhaust container and incubator open ports are configured to maintain an isobar between the reservoir, the exhaust container, and the incubator. In some implementations, at least one of the first incubator open port and the at least one reservoir open port has a filter mounted thereon. In some implementations, the filter mounted on at least one of the first incubator open port and the at least one reservoir open port is a High Efficiency Particulate Air (HEPA) filter. In some implementations, at least one of the second incubator open port and the at least one exhaust container open port has a filter mounted thereon. In some implementations, the filter mounted on at least one of the second incubator open port and the at least one exhaust container open port is a HEPA filter. In some implementations, the system includes an oxygen concentrator in fluid communication with the reservoir cavity for increasing a concentration of oxygen in the reservoir.

In accordance with another aspect, there is provided an incubation system comprising a compressor for providing airflow; a reservoir comprising a reservoir wall defining a reservoir cavity; a reservoir inlet defined in the reservoir wall and being in fluid communication with the entry flow displacement device for receiving the airflow therefrom; and a reservoir outlet defined in the reservoir wall; an incubator comprising an incubator wall defining an incubator cavity, and configured to receive at least one cell culture container therein, wherein the at least one cell culture container is configured to contain a cell culture which produces CO₂ that becomes part of the airflow to create a CO₂ enriched airflow; an incubator inlet defined in the incubator wall and being in fluid communication with the reservoir outlet for receiving the airflow therefrom; an incubator outlet defined in the incubator wall; and an incubator door provided in the incubator wall and having an open position for allowing access to the incubator cavity and a closed position for providing hyperbaric conditions; an exhaust container comprising an exhaust container wall defining an exhaust container cavity; an exhaust container inlet defined in the exhaust container wall and being in fluid communication with the reservoir outlet for receiving the airflow therefrom; and an exhaust container outlet defined in the exhaust container wall; and an exhaust pump in fluid communication with the exhaust container inlet for drawing the CO₂ enriched airflow out of the incubator to maintain a stable pH level for the cell culture.

In some implementations, the pressure in each of the reservoir, incubator and exhaust container is within a range of 1.2 atm to 1.5 atm. In some implementations, the system includes a first heat exchanger downstream of the compressor and configured to reduce a temperature of the pressurized air. In some implementations, the system includes a second heat exchanger downstream of the compressor and configured to reduce or increase a temperature the pressurized air. In some implementations, the system includes at least one filter downstream of the compressor wherein the airflow is filtered prior to air entering the reservoir. In some implementations, the at least one filter comprises a carbon filter. In some implementations, the at least one filter comprises a HEPA filter. In some implementations, the at least one cell culture container comprises: a cell culture container inlet; and a cell culture container outlet, wherein at least one inlet tube connects the cell culture container inlet to the incubator inlet and at least one outlet tube connects the cell culture container outlet to the incubator outlet. In some implementations, the reservoir comprises at least one reservoir open port provided on the wall of the reservoir, the exhaust container comprises at least one exhaust container open port provided on the wall of the exhaust container, the incubator comprises: at least a first incubator open port being in fluid communication with the at least one reservoir open port; and at least a second incubator open port being in fluid communication with the at least one exhaust container open port. In some implementations, each of the reservoir, exhaust container and incubator open ports are configured to actuate in response to a difference in pressure between the reservoir, the exhaust container, and the incubator. In some implementations, each of the reservoir, exhaust container and incubator open ports are configured to maintain an isobar between the reservoir, the exhaust container, and the incubator. In some implementations, at least one of the first incubator open port and the at least one reservoir open port has a filter mounted thereon. In some implementations, the filter mounted on at least one of the first incubator open port and the at least one reservoir open port is a High Efficiency Particulate Air (HEPA) filter. In some implementations, at least one of the second incubator open port and the at least one exhaust container open port has a filter mounted thereon. In some implementations, the filter mounted on at least one of the second incubator open port and the at least one exhaust container open port is a HEPA filter. In some implementations, the system includes an oxygen concentrator in fluid communication with the reservoir cavity for increasing a concentration of oxygen in the reservoir.

In accordance with yet another aspect, there is provided a method of providing cell incubation comprising providing a reservoir, an incubator, and an exhaust container; supplying air at atmospheric pressure to the reservoir; supplying the air from the reservoir to at least one cell culture container located in the incubator for incubation of a cell culture which produces CO₂ that forms a CO₂ enriched air; supplying the CO₂ enriched air from the incubator into the exhaust container to maintain a stable pH for the cell culture; and providing isobaric pressure conditions in the reservoir, the incubator, and the exhaust container.

In accordance with yet another aspect, there is provided a method of providing cell incubation in a hyperbaric environment comprising supplying pressurized air to a hyperbaric chamber in which a reservoir, an incubator, and an exhaust container are located; supplying the pressurized air from the hyperbaric chamber into the reservoir; supplying the pressurized air from the reservoir to at least one cell culture container located in the incubator for incubation of a cell culture which produces CO₂ that forms a CO₂ enriched pressurized air; supplying the CO₂ enriched pressurized air from the incubator into the exhaust container to maintain a stable pH for the cell culture; and providing isobaric pressure conditions in the hyperbaric chamber, the reservoir, the incubator, and the exhaust container.

In accordance with yet another aspect, there is provided a method of providing cell incubation in a hyperbaric environment comprising providing a reservoir, an incubator, and an exhaust container; supplying pressurized air into the reservoir; supplying the pressurized air from the reservoir to at least one cell culture container located in the incubator for incubation of a cell culture which produces CO₂ that forms a CO₂ enriched pressurized air; supplying the CO₂ enriched pressurized air from the incubator into the exhaust container to maintain a stable pH for the cell culture; and providing isobaric pressure conditions in the hyperbaric chamber, the reservoir, the incubator, the at least one cell culture container and the exhaust container.

In some implementations, one of more of the above methods can include one or more features described herein and/or one or more features where the method is performed in a system as described above or herein.

### BRIEF DESCRIPTION OF THE DRAWINGS

FIG. 1 is a diagram of an implementation of a hyperbaric incubation system, which could also be operated at atmospheric conditions.

FIG. 2 is a diagram of another implementation of a hyperbaric incubation system, which could also be operated at atmospheric conditions.

### DETAILED DESCRIPTION

Various aspects of the incubation system and associated methods and its use for incubating cell cultures will be described in further detail below.

The present disclosure relates to systems and methods that facilitate incubation of cell cultures contained within an incubator, such as a hyperbaric incubator, while mitigating issues related to the release and accumulation of CO₂ in a cell culture medium that may increase acidity and kill a culture.

There are various contexts in which hyperbaric conditions may be of interest for incubating cell cultures. Hyperbaric conditions, such as those provided during oxygen therapy, have been found to be beneficial for improved recovery in patients. Providing hyperbaric conditions in cell cultures may provide benefits to the cell cultures as well, such as improved growth rates, and/or experimental conditions that mimic certain real-life conditions of interest. For example, it is envisaged that skin grafts for burn patients may be more efficiently grown using methods and systems of the present disclosure. It may also be advantageous to incubate certain cell cultures in hyperbaric conditions that would mimic real-life conditions for certain organisms and organs such as the lungs.

It may additionally be advantageous to incubate cell cultures at normal atmospheric conditions according to the present disclosure, mimicking real-life conditions for certain organisms or organs such as the lungs, and facilitating CO₂ extraction as well as controlling the concentration of CO₂ and the associated pH level in cell cultures.

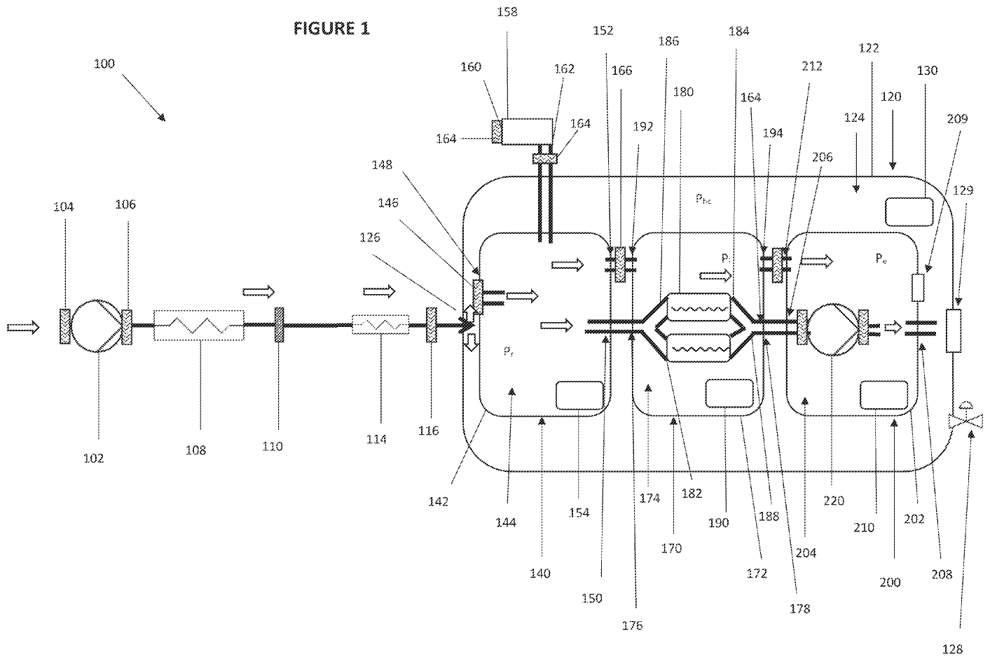

Referring to the drawings, and more particularly to FIG. 1, an incubation system 100 is disclosed. In one implementation, the incubation system 100 comprises a compressor 102, particulate filters 104, 106 mounted upstream and downstream of the compressor 102, a first heat exchanger 108 mounted downstream of the compressor 102, an activated carbon filter 110, a second heat exchanger 114, and a High Efficiency Particulate Air (HEPA) filter 116, which together make up an input assembly. The input assembly can include various components for supplying a pressurized gas, such as air, to the rest of the system. The input assembly is connected to a hyperbaric chamber 120 having therein a reservoir 140, an incubator 170 and an exhaust container 200. As shown by the flow arrows in FIG. 1, air is supplied by the compressor 102 through the input assembly and into the hyperbaric chamber 120. In some implementations, the order of the components may be modified compared to what is shown in FIG. 1 and certain components, such as one or more of the filters and/or heat exchangers, can be absent from the input assembly.

The compressor 102 is operable to increase the pressure in the hyperbaric chamber 120 above atmospheric pressure. In one implementation, the hyperbaric chamber 120 is pressurized to comprise pressurized air at a pressure between 1.2 and 1.6 atm, such as a pressure of about 1.4 atm.

The compressor 102 is configured to receive air under ambient conditions, for example ambient conditions in a laboratory, through a compressor inlet and to pressurize the air so that the air exits through a compressor outlet under hyperbaric conditions. If components presenting air resistance, such as filters, are mounted downstream of the compressor 102, the compressor 102 may be configured to provide air pressure to a level to compensate for these pressure losses. In one implementation, one or more filters, such as illustrated filters 104, 106, carbon filter 110 and HEPA filter 116, may be placed upstream and/or downstream of the compressor 102. This may help, for example, to filter out dust or other particles that may reduce the performance of the incubation environment or impair or influence cell cultures. In one implementation, the one or more filters are configured to additionally filter out microorganisms such as bacteria, mold, and viruses that may be present in the air. In one implementation, one or more of the filters are HEPA rated filters. It is noted that other units could be provided for removing and/or deactivating microorganisms via mechanisms other than filtering. The input assembly is thus configured such that the air supplied to the incubation environment carries no bacteria or molds and is appropriate for the cell culture of interest.

Still referring to FIG. 1, the first heat exchanger 108 is positioned downstream of the compressor 102 and is configured to receive pressurized air from the compressor 102. Air temperature increases as its pressure increases in the compressor 102, and the first heat exchanger 108 is configured to cool this pressurized air. In one implementation, the first heat exchanger 108 may be configured to cool the pressurized air to ambient conditions. In some implementations, the first heat exchanger 108 may be configured to cool or heat the pressurized air to a given target temperature, which can depend on the conditions desired for a particular incubation run.

In some implementations, filters 104, 106 may be positioned both upstream and downstream of the compressor 102. Although the downstream filter 106 has been illustrated as being directly upstream of the first heat exchanger 108, the filter may instead be positioned differently, for example downstream of the first heat exchanger 108. In addition to the filters 104, 106 positioned upstream and downstream of the compressor 102, additional filters may be provided as part of the input assembly. For example, as illustrated in FIG. 1, the carbon filter 110 may be added and configured to filter out volatile organic compounds (VOC's). As illustrated in FIG. 1, HEPA filter 116 may also be positioned in the input assembly. In some implementations, the positioning and type of filters may be changed.

In some implementations, the second heat exchanger 114 may be positioned downstream of the first heat exchanger 108 for further adjusting or regulating the temperature of the pressurized air. In one implementation, the second heat exchanger 114 is a Peltier heat exchanger. A Peltier heat exchanger may be configured to either cool or heat the fluid passing therethrough. Accordingly, a Peltier heat exchanger may allow the temperature of the pressurized air to be increased or decreased, providing greater control and stability over the incubation process. In one implementation, the first heat exchanger 108 may be removed entirely, leaving only the second heat exchanger 114 to regulate the temperature of the pressurized air. In one implementation, the second heat exchanger 114 may be removed entirely, leaving only the first heat exchanger 108. In one implementation, there are no heat exchangers or more than two heat exchangers. In one implementation, as shown in FIG. 1, an additional filter may be positioned downstream of the second heat exchanger 114, further removing contaminants from the pressurized air. In the illustrated implementation, the additional filter is HEPA filter 116.

Temperature regulated pressurized air exits the second heat exchanger 114 and the HEPA filter 116, where applicable, and is supplied to the hyperbaric chamber 120. The hyperbaric chamber 120 comprises a wall 122 defining a cavity 124. The hyperbaric chamber wall 122 has an inlet 126 for receiving the filtered and temperature regulated pressurized air. In one implementation, the hyperbaric chamber wall 122 may be a flexible wall. The hyperbaric chamber 120 further comprises an access opening (not shown in FIG. 1) in the wall 122, which can take the form of a door. The door has an open position to allow a user access inside the hyperbaric chamber 120, and a closed airtight position to allow pressurizing of the hyperbaric chamber 120 to hyperbaric conditions. The door can be configured in various ways. In one implementation, the hyperbaric chamber wall 122 is a flexible wall and the chamber door is a slit with an airtight zipper which extends across part of the wall 122 of the chamber 120. In one implementation, the hyperbaric chamber 120 is a flexible wall hyperbaric chamber such as those used in oxygen therapy, e.g., a Yada™ hyperbaric chamber.

In one implementation, the hyperbaric chamber 120 may comprise an open port 128 which allows for setting the working the pressure of the hyperbaric chamber 120. In one implementation, the open port 128 is a valve which may be closed after the door is closed to allow the hyperbaric chamber pressure to increase to the desired level. In some implementations, and as illustrated in FIG. 1, the open port 128 may be a pressure relief valve configured to open and release pressurized air from inside the hyperbaric chamber 120 to ambient conditions once a certain pressure is reached in order to release the pressurized air from inside the hyperbaric chamber 120 back to ambient conditions. A user may then be able to open the access door to access the inside of the hyperbaric chamber 120. Alternatively, the open port 128 may be an electrically actuated port, configured to open or close in response to a pressure configured by a user and detected by a pressure sensor coupled to the open port 128.

In one implementation, this open port 128 is kept open during incubation, preventing pressurization to hyperbaric conditions, but permitting CO₂ extraction under atmospheric conditions.

The hyperbaric chamber 120 may comprise an additional open port 129, the open port 129 being coupled to a CO₂ sensor. The additional open port 129 may be configured to open when the CO₂ sensor detects an excess amount of CO₂ in the hyperbaric chamber 120. In this configuration, the hyperbaric chamber may be configured to be open or closed to the ambient environment in response to one or both of air pressure and CO₂ concentration.

In one implementation, the hyperbaric chamber 120 may comprise an ultraviolet (UV) light 130 which can be operable, for example, by an external controller, to disinfect the interior of the hyperbaric chamber 120 when a culture is removed and before a new culture is placed therein. The hyperbaric chamber 120 is sized and dimensioned to accommodate the reservoir 140, the incubator 170, and the exhaust container 200 within the cavity. In one implementation, the UV light emits light at a wavelength of 250-280 nm, such as 253 nm.

The reservoir 140 can have a form of a container that receives the pressurized air from the hyperbaric chamber 120. The reservoir 140 comprises a reservoir wall 142 defining a cavity 144 for receiving the pressurized air. The reservoir wall 142 comprises an inlet 146 defined therein and in fluid communication with the hyperbaric chamber 120 for receiving the pressurized air therefrom. The inlet 146 may have a filter 148 mounted thereon. In one implementation, the filter 148 is a HEPA filter. The reservoir 140 also comprises an outlet 150 defined in the wall 142, shown mounted opposite to the inlet 146 in FIG. 1, and in fluid communication with cell culture containers 180 for supplying the pressurized air to the cell culture containers 180. In one implementation, the reservoir 140 additionally comprises a UV light 154 which can be operable, for example, by an external controller, to disinfect the interior of the reservoir 140. The reservoir 140 may additionally have an access opening having a door to allow, for example, to change the UV light 154, where applicable.

In one implementation, an oxygen concentrator 158 may additionally be coupled to be in fluid communication with the reservoir cavity 144. An oxygen concentrator is a device that removes nitrogen from the air it receives. Given that ambient air is mostly composed of nitrogen and oxygen, an oxygen concentrator removes the nitrogen to provide an oxygen rich output airflow. In one implementation, the oxygen concentrator 158 is mounted outside the hyperbaric chamber 120 with air entering through inlet 160 from outside the hyperbaric chamber 120 and entering the reservoir 140 via outlet tubes 162 connected to the reservoir 140. In one implementation, the outlet tubes 162 are tygon tubes. The tubes 162 may have a diameter between 7-10 mm. Alternatively, they may have any other desired diameter. The oxygen concentrator draws air from the outside and discharges oxygen-enriched air to the reservoir 140. In one implementation, filters 164 are positioned on an outlet and an inlet of the oxygen concentrator. In one implementation, filters 164 are HEPA filters. Alternatively, if oxygen rich air is not required, the oxygen concentrator may be removed entirely.

The reservoir 140 further comprises an open port 152 provided on the reservoir wall. Open ports are mounted on the reservoir 140, incubator 170 and exhaust compartment 200 and configured to maintain the reservoir 140, incubator 170 and exhaust compartment 200 in fluid communication with one another. Open ports allow pressure equalization between said compartments, so that isobaric conditions may be maintained between the reservoir 140, incubator 170 and exhaust compartment 200. In one implementation, the calibrated port is an electrically actuated port. The open ports are calibrated to open or close depending on the desired pressure in the reservoir 140, incubator 170 and exhaust compartment 200. For example, the open ports may be configured to receive pressure readings from pressure gauges and to open or close in response. The open ports thus permit pressure regulation across the reservoir 140, incubator 170 and exhaust compartment 200 by selectively opening or closing. The open port 152 may have a filter 166 mounted thereon. In one implementation, the filter 166 is a HEPA filter.

The incubator 170 can have a form of a cabinet that receives the pressurized air from the reservoir 140. The incubator 170 comprises an incubator wall 172 defining an incubator cavity 174. Cell culture containers 180 may be received in the incubator cavity 174 for allowing cell cultures to metabolize and reproduce in the cell culture containers 180. The incubator 170 is a container configured to maintain temperature and humidity to encourage growth of the cell cultures. In one implementation, the incubator 170 is a so-called CO₂ incubator, such as those sold by Thermo Fisher Scientific™. In one implementation, the CO₂ incubator resembles a small refrigerator with a metal housing having heating elements, at least one shelf for receiving cell culture containers 180 thereon, and an access opening comprising a door for allowing a user access to the cell culture containers 180 (for example, for removing and replacing the cell culture containers 180). A water container configured to receive water may additionally be included or added to the incubator 170 to ensure that a desired level of humidity is maintained in the incubator 170. In one implementation, the incubator 170 may be a water-jacketed incubator. A water-jacketed incubator may comprise a double wall housing with water flowing between the walls. A water-jacketed incubator may be more temperature stable than a CO₂ incubator without a water jacket due to the higher specific heat capacity of water.

The incubator 170 comprises an incubator inlet 176 defined in the incubator wall 172 and in fluid communication with the reservoir outlet 150. Accordingly, pressurized air from the reservoir 140 is supplied to the incubator 170 through the incubator inlet 176. The incubator 170 additionally comprises an incubator outlet 178 defined in the incubator wall 172 opposite to the incubator inlet 176 and in fluid communication with the exhaust container 200. The incubator 170 additionally comprises an access opening in the incubator wall 172 having a door. The door has an open position to allow access to the incubator cavity 174 and a closed position for sealing the incubator cavity 174. Typically, a user may open the door to monitor, place, replace or remove cell culture containers 180 from within the incubator 170. Once the user has manipulated the cell culture containers 180, the door is closed so that the conditions in the incubator 170 may be regulated as desired, for example to a specific pressure, temperature, or humidity. As the user opens and closes the incubator 170, the incubator 170 may be prone to contaminants entering. In one implementation, the reservoir 140 additionally comprises a UV light 190 which can be operable, for example by an external controller, to disinfect the interior of the incubator 170 between cell cultures.

The cell culture containers 180 received in the incubator 170 can each be configured to comprise an inlet 182 and an outlet 184. In one implementation, the cell culture container inlet 182 may be connected to the incubator inlet 176 through an inlet tube 186 and the cell culture container outlet 184 may be connected to the incubator outlet 178 through an outlet tube 188. In one implementation, the tubes are Tygon tubes having a diameter between 7-10 mm. The tubes 186, 188 can provide the benefit of providing fresh and sterilized air from the reservoir 140, preventing potentially contaminated air from inside of the incubator 170 as a result of opening the incubator door to manipulate the cell culture containers 180. In the implementation illustrated in FIG. 1, the tubes 186, 188 may have a main artery which branches out to inlets 182 and outlets 184 of the cell culture containers 180, where there is a plurality of cell culture containers.

In one implementation, the inlet 182 and outlet 184 of each cell culture container 180 may comprise a disposable syringe-type HEPA filter sized and dimensioned to be mounted onto the inlet 182 and outlet 184 of the cell culture containers 180.

In one implementation, the cell culture container 180 is composed of a light polymer material. In one implementation, the cell culture container 180 has a filter at the cell culture container inlet 182 and another filter at the cell culture container outlet 184. In one implementation, the air/cell culture medium ratio is 1:1 or higher, that is to say, the volume of air is the same or greater than the volume of the cell culture medium, such as a liquid. A 1:1 ratio or greater ratio between air and the cell culture medium may improve passive diffusion by providing a greater amount of air.

The cell cultures in the cell culture containers 180 are configured to receive the pressurized (and filtered) air from the reservoir 140 through the incubator inlet 176, to metabolize and reproduce, and in so doing can produce CO₂. The cell culture containers 180 are also configured to be in isobar with the air inside the incubator 170. The produced CO₂ becomes part of the pressurized air to create CO₂ enriched pressurized air, or polluted air. The CO₂ enriched pressurized air is subsequently removed through the incubator outlet 178.

In one implementation, the incubator 170 is an insulated incubator (including a water-jacketed incubator) to reduce temperature fluctuations in the incubator cavity. The incubator 170 further comprises a first open port 192 and a second open port 194 provided on the incubator wall 172.

The exhaust container 200 is configured to be in isobar with the incubator 170, and to receive the CO₂ enriched pressurized air from the cell culture containers 180 in the incubator 170. The exhaust container 200 comprises an exhaust container wall 202 defining an exhaust container cavity 204. The exhaust container wall 202 comprises an inlet 206 defined thereon and in fluid communication with the cell culture containers 180 in the incubator 170 for receiving the CO₂ enriched pressurized air therefrom.

The exhaust container 200 also comprises an outlet 208 defined in the wall 202 and in fluid communication with the hyperbaric chamber 120 for supplying the CO₂ enriched pressurized air thereto. The outlet 208 may be coupled to a pressure sensor to open or close in response to pressure inside the exhaust container 200. The exhaust container 200 may also comprise an open port 209 coupled to a CO₂ sensor. The open port 209 may be configured to open when the CO₂ sensor detects an excess amount of CO₂ in the exhaust container 200. In this configuration, the exhaust container 200 may be configured to be open or closed to the ambient environment in response to one or both of air pressure and CO₂ concentration.

In one implementation, the exhaust container 200 additionally comprises a UV light 210 which can be operable, for example, by an external controller, to disinfect the interior of the exhaust container 200. In one implementation, the UV lights 130, 154, 190, 210 of the hyperbaric chamber 120, the reservoir 140, the incubator 170, and the exhaust container 200 can be run for a total of five minutes to disinfect their respective interior surfaces prior to starting a new culture. They may additionally be run throughout the incubation. The exhaust container 200 may additionally comprise an access opening having a door thereon for providing access to the interior components.

The exhaust container 200 further comprises an open port 212 provided on the exhaust container wall 202. The open port 212 is in fluid communication with the second open port 194 on the incubator wall 172, allowing fluid communication between the exhaust container 200 and the incubator 170. The open ports 152, 192, 194, 212 on the reservoir 140, the incubator 170 and the exhaust container 200 may be operable to open and close in response to pressure fluctuation across the reservoir 140, incubator 170, and exhaust container 200. That is to say, the open ports 152, 192, 194, 212 may be configured to ensure isobaric conditions across the reservoir 140, the incubator 170, and the exhaust container 200 and to mitigate pressure fluctuations across said compartments.

The hyperbaric chamber 120 is also isobaric with the reservoir 140, the incubator 170, and the exhaust container 200, such that the pressure P_{hc} of the hyperbaric chamber 120 is equal to pressure Pᵣ of the reservoir 140, pressure Pᵢ of the incubator 170, and pressure Pₑ of the exhaust container 200. At least one of the hyperbaric chamber 120, reservoir 140, incubator 170, and exhaust container 200 may have a pressure gauge to allow a user to determine the pressure in the respective compartment. In one implementation, the compressor 102 may be computer controlled and configured to increase or reduce flow to regulate the pressure in response to pressure readings from the hyperbaric chamber 120, reservoir 140, incubator 170 and/or exhaust container 200.

In some implementations, an exhaust pump 220 is positioned in the exhaust container 200. The exhaust pump 220 is in fluid communication with the inlet 206 of the exhaust container 200 for drawing air from the cell culture containers 180 in the incubator 170. The exhaust pump 220 is configured to draw air from the cell culture containers 180, in effect mimicking exhalation, to regulate the CO₂ present in the cell culture containers 180. In one implementation, the exhaust pump 220 is a centrifugal pump. The exhaust pump 220 is configured to exhaust out the CO₂ enriched pressurized air into the exhaust container 200. The exhausted CO₂ enriched pressurized air is supplied into the hyperbaric chamber 120 through the exhaust container outlet 208, which may additionally have a filter mounted thereon. The exhausted CO₂ enriched pressurized air may then circulate in the hyperbaric chamber 120 or exit the hyperbaric chamber 120 through the hyperbaric chamber valve 128. The hyperbaric chamber valve 128 may therefore release pressure prior to opening the door to the hyperbaric chamber 120.

The combined effect of the open ports 152, 192, 194, 212 across the reservoir 140, incubator 170 and exhaust container 200 is to maintain an isobaric condition between the said containers and with the cell culture containers 180, thus preventing any pressure differential that could cause violent rupture of the cell culture containers 180. It is accordingly possible to maintain a sterile and constant flow of air through the hyperbaric incubation system 100 as described. Although three separate compartments (reservoir 140, incubator 170, exhaust container 200) have been illustrated, it is envisaged that in some implementations the user may instead use two compartments. For example, the exhaust container 200 may be removed, and the exhaust pump may instead be positioned in the hyperbaric chamber 120. Alternatively, both the exhaust container 200 and the reservoir 200 may be removed, so that only the incubator 170 is positioned inside the hyperbaric chamber 120. Additionally, although the hyperbaric chamber 120 is configured to receive the reservoir 140, incubator 170 and the exhaust container 200 therein to effectively maintain a null pressure differential between the interior and exterior of each of these compartments, it will also be possible to run the incubation system 100 under normal atmospheric conditions. The closed-circuit fresh air circulation would then make it possible to control cell culture pH with less pH balancing compounds, such as buffer salts.

In accordance with the above, a method of starting up the hyperbaric incubation will now be described. Once the hyperbaric incubation system 100 is prepared, and prior to placing the cell cultures in the incubator 170 as illustrated for example in FIG. 1, the user may operate the UV lights 130, 154, 190, 210 of each of the compartments 120, 140, 170, 200 to sterilize the interior of each compartment. In one implementation, the UV lights 130, 154, 190, 210 are operated for five minutes each to provide adequate sterilization. Cell culture containers 180 are then placed within the incubator 170. Tubes such as tubes 186, 188 may be used to connect the cell culture container inlets 182 and outlets 184 to the incubator inlet 176 (connected to the cavity 144 of the reservoir 140) and outlet 178 (connected to the cavity 204 of the exhaust container 200). The incubator door is then closed and the incubator 170 allowed to regulate the temperature and humidity therein. The valve 128 of the hyperbaric chamber 120 and the door are closed to seal the hyperbaric chamber 120. The compressor 102 is then started to pressurize the hyperbaric chamber 120 (and the compartments 140, 170, 200 therein). The pressurized air passes by any filters and/or heat exchangers to provide temperature controlled filtered air to the hyperbaric chamber 120. Once the interior pressure of the hyperbaric chamber 120 reaches the desired pressure, the flow of the compressor 102 may be reduced to a level needed to maintain the pressure while reducing air movement and potential associated disturbances. In one implementation, the pressure in the hyperbaric chamber 120 may be reached in about ten minutes. In one implementation, the compressor 102 may begin pressurizing at hundreds of liters per minute, and ramp down to tens of liters per minute as the pressure increases. The valve 128 on the hyperbaric chamber 120 may additionally allow any excess air to escape into the ambient. Once the isobaric condition has been satisfied between the compartments, the exhaust pump 220 may be activated to start drawing CO₂ enriched pressurized air from the cell culture containers 180. The flowrate of the exhaust pump 220 may be adjusted as a function of the amount of CO₂ enriched pressurized air that needs to be removed to maintain the pH level of the cell culture containers 180 within the desired range.

If access is required to the cell culture containers 180 or the interior of the hyperbaric chamber 120, the compressor 102 may be stopped. The valve 128 on the hyperbaric chamber 120 may be opened to allow the hyperbaric chamber 120 to depressurize to atmospheric condition. In one implementation, the depressurization process takes approximately ten minutes. Once the incubation system 100 is depressurized, the user may access the interior of the hyperbaric chamber 120 or the cell culture containers within the incubator 170.

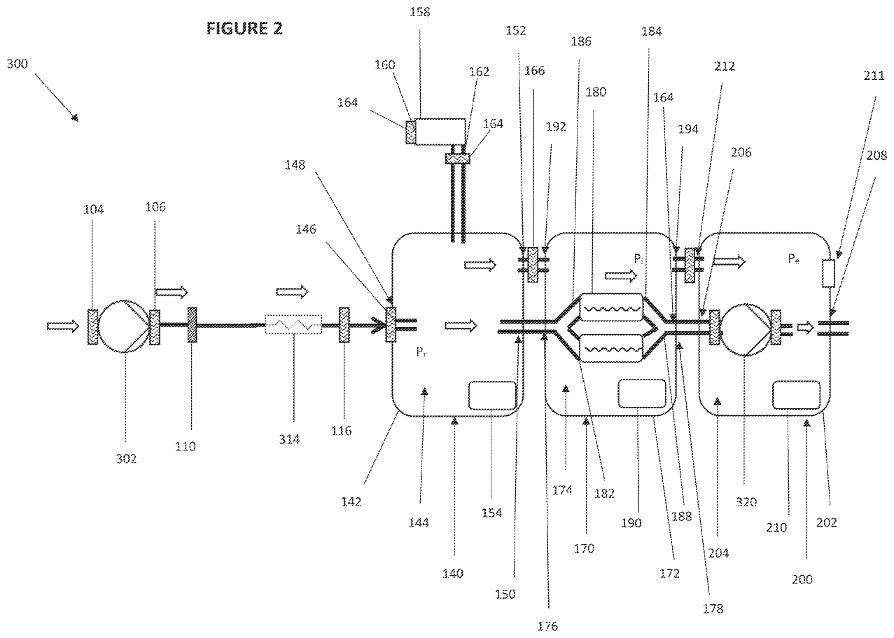

In accordance with another aspect and as illustrated in FIG. 2, there is provided an incubation system 300 operating at hyperbaric conditions while placed in normal atmospheric conditions. The atmospheric incubation system 300 of FIG. 2 is similar to the hyperbaric incubation system 100 of FIG. 1, with the exception that there is no hyperbaric chamber 120 since the cabinets are strong enough to maintain the air pressure inside. For example, the body of each of the reservoir 140, incubator 170 and exhaust compartment 200 may be reinforced. In this configuration, the hyperbaric chamber may be omitted as each of the reservoir 140, incubator 170 and exhaust compartment 200 is a standalone hyperbaric component. In the illustrated implementation of FIG. 2, there is a compressor 302 which is configured to supply air from the ambient to the reservoir 140. Similarly to previously described incubation system 100, airflow emanating from the compressor 302 is supplied to an input assembly comprising at least one filter, such as particulate, charcoal and/or HEPA filters. The input assembly may further comprise a heat exchanger 314 configured to either cool or heat the fluid passing therethrough. The heat exchanger 314 may, in one implementation, be a Peltier heat exchanger.

The incubation system 300 further comprises an exhaust pump 320 positioned in the exhaust container 200 and configured to draw CO₂ enriched airflow out of the cell culture containers 180 in the incubator 170 to maintain a stable pH level for the cell culture. Similar to the exhaust pump 220, the exhaust pump 320 helps to mimic exhalation to regulate the CO₂ present in (and therefore the acidity of) the cell culture containers 180. The CO₂ enriched airflow is then exhausted to the exhaust container 200, which allows the CO₂ enriched airflow to exit the incubation system 300 via the outlet 208 into the ambient. In one implementation the outlet 208 may be an electrically actuated port, configured to open or close in response to a pressure configured by a user and detected by a pressure sensor coupled to the open port 128.

In one implementation, a valve 211 may be mounted to the outlet 208 to allow a user to manually release pressure when the exhaust container 200 has a pressure different to the exterior of the exhaust container 200, for example when the exhaust container 200 is hyperbaric while the surrounding environment is under atmospheric pressure. The valve 211 therefore allows the user to release pressure, allowing the user to open the access door of the exhaust container 200.

The incubation system 300 provides stronger cabinets for the reservoir 140, incubator 170, and exhaust container 200 without the need for a hyperbaric chamber, allowing each of the reservoir 140, incubator 170, and exhaust container 200 to maintain the hyperbaric inside without rupture of their structures. The reservoir 140, incubator 170 and exhaust container 200 are configured to withstand a pressure differential between their respective interior and exterior. They may additionally resist fluid leakage at a given pressure differential. There is accordingly a need for a pressure release valve on each of the containers to permit safely opening the door to the respective container, as well as the open ports 152, 192, 194, 212 to maintain an isobar between the reservoir 140, incubator 170 and exhaust container 200. It is envisaged that the incubation system 300 may also be run at atmospheric conditions.

The embodiments described in the present disclosure provide multiple benefits. In a gas mixture, the total pressure of the mixture is equal to the sum of the partial pressures of its constituent components. Accordingly, an increase in pressure from atmospheric to hyperbaric conditions will lead to an increase in the partial pressure of CO₂ in the gas mixture. The pH level of a cell culture is related to the partial pressure of CO₂ in the cell culture environment. Accordingly, providing hyperbaric conditions to a cell culture without regulating the amount of CO₂ in the gas mixture will lead to a reduction in pH level, creating greater acidity in the cell culture environment. If the pH level of the cell culture is not precisely controlled, the cell culture may die. For example, the pH level in a cell culture may need to be controlled within a range of 7.3-7.6 to maintain the cell culture in ideal conditions.

The human body regulates the pH level of its blood by regular breathing. It is, possible, however, to vary the pH of blood by changing our breathing pattern. As an example, hyperventilating wherein a person rapidly inhales air, increases the pH level of blood making it more alkaline. By contrast, inhaling air from a small, closed container, such as a paper bag, will result in the person inhaling CO₂ that had previously been exhaled. This increases the amount of CO₂ in the blood, reducing its pH level making it more acidic. It has been noted that this additional acidity may ultimately to death of the cell culture.

It is therefore noted that a person changing their rate of respiration may change their blood pH level by changing the concentration of CO₂ in their blood. It is an object of the present disclosure as set out above to artificially replicate this process in an incubation system to regulate the level of CO₂ in cell cultures within the incubation system by use of a CO₂ extraction and incubation method or an incubator as described herein.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A hyperbaric incubation system comprising:
a compressor for providing pressurized air;
a hyperbaric chamber comprising:
a hyperbaric chamber wall defining a hyperbaric chamber cavity;
a hyperbaric chamber inlet defined in the wall for receiving the pressurized air from the compressor;
a hyperbaric chamber pressure relief open port defined in the wall, wherein closing the hyperbaric chamber pressure relief open port allows hyperbaric chamber pressurization and opening the hyperbaric chamber pressure relief open port allows hyperbaric chamber depressurization; and
a hyperbaric chamber door provided in the hyperbaric chamber wall and having an open position for allowing access to the hyperbaric chamber cavity and a closed position for providing hyperbaric conditions;
a reservoir located within the hyperbaric chamber cavity and comprising:
a reservoir wall defining a reservoir cavity;
a reservoir inlet defined in the reservoir wall and being in fluid communication with the hyperbaric chamber cavity for receiving the pressurized air therefrom; and
a reservoir outlet defined in the reservoir wall;
an incubator located within the hyperbaric chamber cavity and comprising:
an incubator wall defining an incubator cavity, and configured to receive at least one cell culture container therein, wherein the at least one cell culture container is configured to contain a cell culture which produces CO₂ that becomes part of the pressurized air to create CO₂ enriched pressurized air;
an incubator inlet defined in the incubator wall and being in fluid communication with the reservoir outlet for receiving the pressurized air therefrom;
an incubator outlet defined in the incubator wall; and
an incubator door provided in the incubator wall and having an open position for allowing access to the incubator cavity and a closed position for providing hyperbaric conditions;
an exhaust container located within the hyperbaric chamber cavity and comprising:
an exhaust container wall defining an exhaust container cavity;
an exhaust container inlet defined in the exhaust container wall and being in fluid communication with the reservoir outlet for receiving the pressurized air therefrom; and
an exhaust container outlet defined in the exhaust container wall and being in fluid communication with the hyperbaric chamber cavity; and
an exhaust pump in fluid communication with the exhaust container inlet for drawing the CO₂ enriched pressurized air out of the incubator to maintain a stable pH for the cell culture.

2. The hyperbaric incubation system of claim 1, wherein pressure of the hyperbaric incubation system is within a range of 1.2 atm to 1.5 atm.

3. The incubation system of claim 1, further comprising a first heat exchanger downstream of the compressor and configured to reduce a temperature of the pressurized air.

4. The incubation system of claim 1, further comprising a second heat exchanger downstream of the compressor and configured to reduce or increase a temperature the pressurized air.

5. The incubation system of claim 4, wherein the second heat exchanger is a Peltier heat exchanger.

6. The incubation system of claim 1, further comprising at least one filter downstream of the compressor wherein the pressurized air is filtered prior to air entering the hyperbaric chamber.

7. The incubation system of claim 6, wherein the at least one filter comprises a carbon filter.

8. The incubation system of claim 6, wherein the at least one filter comprises a HEPA filter.

9. The incubation system of claim 1, wherein the at least one cell culture container comprises:
a cell culture container inlet; and
a cell culture container outlet,
wherein at least one inlet tube connects the cell culture container inlet to the incubator inlet and at least one outlet tube connects the cell culture container outlet to the incubator outlet.

10. The incubation system of claim 1, wherein:
the reservoir comprises at least one reservoir open port provided on the wall of the reservoir,
the exhaust container comprises at least one exhaust open port provided on the wall of the exhaust container,
the incubator comprises:
at least a first incubator open port being in fluid communication with the at least one reservoir open port; and
at least a second incubator open port being in fluid communication with the at least one exhaust container open port.

11. The incubation system of claim 10, wherein each of the reservoir, exhaust container and incubator open ports are configured to actuate in response to a difference in pressure between the reservoir, the exhaust container, and the incubator.

12. The incubation system of claim 11, wherein each of the reservoir, exhaust container and incubator open ports are configured to maintain an isobar between the reservoir, the exhaust container, and the incubator.

13. The incubation system of claim 10, wherein at least one of the first incubator open port and the at least one reservoir open port has a filter mounted thereon.

14. The incubation system of claim 13, wherein the filter mounted on at least one of the first incubator open port and the at least one reservoir open port is a High Efficiency Particulate Air (HEPA) filter.

15. The incubation system of claim 10, wherein at least one of the second incubator open port and the at least one exhaust container open port has a filter mounted thereon.

16. The incubation system of claim 15, wherein the filter mounted on at least one of the second incubator open port and the at least one exhaust container open port is a HEPA filter.

17. The incubation system of claim 1, further comprising an oxygen concentrator in fluid communication with the reservoir cavity for increasing a concentration of oxygen in the reservoir.

18. An incubation system comprising:
a compressor for providing airflow;
a reservoir comprising:
a reservoir wall defining a reservoir cavity;
a reservoir inlet defined in the reservoir wall and being in fluid communication with the entry flow displacement device for receiving the airflow therefrom; and
a reservoir outlet defined in the reservoir wall;
an incubator comprising:
an incubator wall defining an incubator cavity, and configured to receive at least one cell culture container therein, wherein the at least one cell culture container is configured to contain a cell culture which produces CO₂ that becomes part of the airflow to create a CO₂ enriched airflow;
an incubator inlet defined in the incubator wall and being in fluid communication with the reservoir outlet for receiving the airflow therefrom;
an incubator outlet defined in the incubator wall; and
an incubator door provided in the incubator wall and having an open position for allowing access to the incubator cavity and a closed position for providing hyperbaric conditions;
an exhaust container comprising:
an exhaust container wall defining an exhaust container cavity;
an exhaust container inlet defined in the exhaust container wall and being in fluid communication with the reservoir outlet for receiving the airflow therefrom; and
an exhaust container outlet defined in the exhaust container wall; and
an exhaust pump in fluid communication with the exhaust container inlet for drawing the CO₂ enriched airflow out of the incubator to maintain a stable pH level for the cell culture.

19. The hyperbaric incubation system of claim 18, wherein pressure in each of the reservoir, incubator and exhaust container is within a range of 1.2 atm to 1.5 atm.

20. The incubation system of claim 18, further comprising a first heat exchanger downstream of the compressor and configured to reduce a temperature of the pressurized air; a second heat exchanger downstream of the compressor and configured to reduce or increase a temperature the pressurized air; at least one filter downstream of the compressor wherein the airflow is filtered prior to air entering the reservoir; wherein the at least one cell culture container comprises a cell culture container inlet, a cell culture container outlet, wherein at least one inlet tube connects the cell culture container inlet to the incubator inlet and at least one outlet tube connects the cell culture container outlet to the incubator outlet; wherein the reservoir comprises at least one reservoir open port provided on the wall of the reservoir, the exhaust container comprises at least one exhaust container open port provided on the wall of the exhaust container, the incubator comprises at least a first incubator open port being in fluid communication with the at least one reservoir open port and at least a second incubator open port being in fluid communication with the at least one exhaust container open port; wherein each of the reservoir, exhaust container and incubator open ports are configured to (i) actuate in response to a difference in pressure between the reservoir, the exhaust container, and the incubator and (ii) maintain an isobar between the reservoir, the exhaust container, and the incubator.
